(19) United States
(12) Patent Application Publication
(10) Pub. No.: US 2017/0368049 A1
(43) Pub. Date: Dec. 28, 2017

(54) COMPOSITE CAPSULES COMPRISING RALOXIFENE, AND VITAMIN D OR ITS DERIVATIVES

(71) Applicant: HANMI PHARM. CO., LTD., Hwaseong-si, Gyeonggi-do (KR)

(72) Inventors: Young Hun KIM, Suwon-si (KR); Taek Kwan KWON, Suwon-si (KR); Jin Young PARK, Seoul (KR); Seung Bin YOUN, Hwaseong-si (KR); Jae Hyun PARK, Suwon-si (KR); Jong Soo WOO, Suwon-si (KR); Yong Il KIM, Suwon-si (KR)

(73) Assignee: HANMI PHARM. CO., LTD., Hwaseong-si, Gyeonggi-do (KR)

(21) Appl. No.: 15/546,715

(22) PCT Filed: Jan. 28, 2016
(86) PCT No.: PCT/KR2016/000959

§ 371 Date: Jul. 27, 2017

(30) Foreign Application Priority Data
Jan. 28, 2015 (KR) 10-2015-0013537

(51) Int. Cl. A61K 31/4535; A61K 9/20; A61K 9/16; A61K 31/593; A61K 9/48

(52) U.S. Cl. CPC A61K 31/4535; A61K 31/593; A61K 9/4808; A61K 9/1652; A61K 9/2054; A61K 9/2086

(57) ABSTRACT

A composite capsule and a method of preparing the same are provided. The composite capsule includes: a raloxifene separate layer including raloxifene or a pharmaceutically acceptable salt thereof; and a vitamin D separate layer including vitamin D or a derivative thereof, wherein the raloxifene separate layer and the vitamin D separate layer are separated from one another in the composite capsule.

### TECHNICAL FIELD

The present disclosure relates to a composite capsule including raloxifene or a pharmaceutically acceptable salt thereof, and vitamin D or its derivative, the composite capsule having improved patient's compliance and improved stability of the active ingredients, and a method of preparing the composite capsule.

### BACKGROUND ART

Osteoporosis is a skeletal disorder pathologically characterized by absolutely decreased bone resorption, known to be induced by overall bone loss due to imbalance in osteogenesis. Bone mineral density (BMD) as a main determination factor of osteoporosis reaches a maximum level at the age of 20s, and decreases gradually afterward but sharply in post-menopause. Rapid reduction in postmenopause is attributed to loss of calcium balance caused by increased calcium loss from postmenopausal estrogen deficiency, reduced intestinal calcium absorption, insufficient calcium intake, and the like. Hormone replacement therapy (HRT) has been studied for the treatment of such osteoporosis.

Administration of estrogen to menopausal women may prevent osteoporosis and cardiovascular diseases, and consequentially lead to life extension. As a matter of fact, however, many women still do not receive HRT or mostly stop the HRT in less one year of the treatment. Main reasons for the discontinuation of HRT are anxiety about breast cancer and onset of uterine bleeding. Therefore, there is a need for the development of ideal estrogen formulations that are good for bones and cardiovascular systems and does not affect on breast and uterine.

Raloxifene, a class of selective estrogen receptor modulator (SERM) drugs, is known to prevent osteoporosis and cardiovascular diseases consequentially to extend life span, with inhibitory effects on growth of endometrial tissue and breast epithelial tissue and lowering lipid level (V. Craig Jordan, Nature Reviews Cancer 7, 46-53, (2007)) Raloxifene with the chemical name of 6-hydroxy-2-(4-hydroxyphenyl)-3-[4-(2-piperidinoethoxy)benzoyl]benzo[b]thiophene may be prepared using any of the methods disclosed in U.S. Pat. No. 4,133,814, U.S. Pat. No. 4,418,068 or U.S. Pat. No. 4,380,635.

Raloxifene is effective as a preventive and therapeutic agent for osteoporosis in postmenopausal women. Evista® tablet (marketed by Eli Lilly and Co.) including raloxifene hydrochloride as an active ingredient is currently available on the market as a preventive and therapeutic agent for osteoporosis, and it was approved to add an indication related to invasive breast cancers by the US Food and Drug Administration (FDA) in 2007.

According to the FDA's approval data related to Evista, Evista is found to be effective for bone mineral density (BMD) improvement, and in particular, for improving the rate of increase in bone mineral density and the bone mineral densities of spine, femoral neck, lumbar spine, and coxal articulation, but not to be effective for non-vertebral fracture (JAMA, 282, 637-645, 3, (1999)). However, Evista can be taken without meal and thus have improved patient's compliance. Furthermore, Evista may lower the risk of developing breast cancer in high-risk patients for postmenopausal breast cancer, and its sales are on sharply upward trend.

In treating osteoporosis with a SERM drug, it is recommended to supplement cholecalciferol usually by about 400 IU to about 1,000 IU a day. Cholecalciferol is generated from 7-dehydrocholesterol in the skin by ultraviolet ray irradiation, and converted via metabolism into calciferol in the liver and then into calcitriol in the kidneys to represent the main activities. Cholecalciferol is known to improve bone mineral density and not only vertebral but also non-vertebral fractures. Due to such a complementary therapeutic effect of a SERM drug with cholecalciferol, coprescription of raloxifene and cholecalciferol is currently prevalent, which is also introduced in academic literature (G. Bovin, et al., The Journal of Clinical Endocrinology & Metabolism 88(9), 4199-4205 (2003)). However, research on a composite formulation including the two drugs, i.e., raloxifene and cholecalciferol, in a single unit dosage form has not been reported yet. It also has been difficult to develop a composite formulation of raloxifene and cholecalciferol due to reduced stability resulting from interactivity between the two active ingredients.

Raloxifene is unstable when exposed, for forced degradation, to acid, basic, or light, and may produce breakdown products, in particular under oxidation conditions. N-oxide, 6-hydroxy-2-(4-hydroxyphenyl)benzo[b]thiophen-3-yl-]-[4-(2-piperdin-l-yl-ethoxy)phenyl]methanone as a breakdown product of raloxifene is increased under such unstable conditions, and the amount of raloxifene may be reduced by about 2.5% when exposed to a 3% hydrogen peroxide solution at room temperature for about 30 minutes (G. Sowjanya et al., Journal of Delivery & Therapeutics 2(4), 175-181 (2012)).

Cholecalciferol (vitamin D3) also has poor physiochemical stability and may not ensure stability with time. Generally, cholecalciferol is known to be susceptible to moisture, light, and heat. As a result of a photoreaction test with cholecalciferol, trans-vitamin D3 (Formula 1) was found to be increased. The amount of trans-vitamin D₃ (after storage in a photostability chamber under 1,200,000 LUX for 18.44 hours) was increased by about 1.2% with respect to initial level, while the amount of vitamin D₃ was reduced by about 10%.

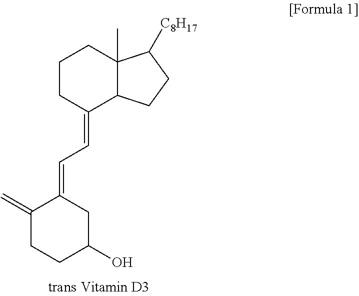

### DETAILED DESCRIPTION OF THE INVENTION

### Technical Problem

The present disclosure provides an oral composite formulation including raloxifene, and vitamin D or a derivative thereof in a single unit dosage form, the oral composite formulation having improved medication compliance of a patent who requires co-administration of raloxifene, and vitamin D or a derivative thereof, without stability reduction caused from interactivity between raloxifene, and vitamin D or a derivative thereof.

The present disclosure provides a method of preparing the composite oral formulation including raloxifene, and vitamin D or a derivative thereof.

### Technical Solution

According to an aspect of the present invention, there is provided a composite capsule including: a raloxifene separate layer including raloxifene or a pharmaceutically acceptable salt thereof; and a vitamin D separate layer including vitamin D or a derivative thereof, wherein the raloxifene separate layer and the vitamin D separate layer are separated from one another in the capsule.

According to another aspect of the present invention, there is provided a method of preparing the above-described composite capsule, the method including: forming a mixture including raloxifene or a pharmaceutically acceptable thereof, and a pharmaceutically acceptable additive into granules or tablets; forming a mixture including vitamin D or a derivative thereof, and a pharmaceutically acceptable additive into granules or tablets; and filling a hard capsule with the granules or tablets of raloxifene and the granules or tablets of vitamin D or a derivative thereof to form a raloxifene separate layer and a vitamin D separated layer.

### Advantageous Effects

According to the one or more embodiments of the present disclosure, a composite capsule may include raloxifene or a pharmaceutically acceptable salt thereof, and vitamin D or a derivative thereof as separate layers in the capsule, to thereby block interaction between the two active ingredients. In other words, the composite capsule may include the two active ingredients, for example, raloxifene, and vitamin D or a derivative thereof, in a single unit dosage form, and thus may have improved patient's compliance. Nearly no stability reduction caused from interaction between the active ingredients may occur in the composite capsule, and thus the composite capsule may have stable efficacy with improved stability over time of the active ingredients. Furthermore, due to a complementary therapeutic effect of the active ingredients, the composite capsule may be used for the effective treatment of osteoporosis, including both vertebral fracture and non-vertebral fracture.

### BRIEF DESCRIPTION OF THE DRAWINGS

FIG. 1 is a schematic view of a composite capsule of Example 1;

FIG. 2 is a schematic view of a composite capsule of Example 4;

FIG. 3 is a schematic view of a composite capsule of Example 5; and

FIG. 4 is a graph illustrating the results of a raloxifene dissolution test with respect to time of the composite formulations of Examples 1 and 6 to 8.

### MODE FOR INVENTION

Unless otherwise defined, all terms (including technical and scientific terms) used herein have the same meaning as commonly understood by one of ordinary skill in the art to which this invention belongs. Although exemplary methods or materials are listed herein, other similar or equivalent ones are also within the scope of the present invention. All publications disclosed as references herein are incorporated in their entirety by reference.

An aspect of the present disclosure provides a composite capsule comprising: a raloxifene separate layer including raloxifene or a pharmaceutically acceptable salt thereof; and a vitamin D separate layer including vitamin D or a derivative thereof, wherein the raloxifene separate layer and the vitamin D separate layer are separated from one another in the composite capsule.

As used herein, the term “separate layer” refers to a layer containing a single active pharmaceutical ingredient, separated from other active pharmaceutical ingredients. The separate layer may not be a continuous layer. The separate layer may be a discontinuous layer of a plurality of granules in which a plurality of different active pharmaceutical ingredients are not intermingled.

As used herein, the expression “separated from one another” refers to the state where a plurality of active ingredients are separated from one another not to interact with each other during storage of the composite formulation.

The raloxifene separate layer and the vitamin D separate layer may each independently be in the form of granules or tablets. The raloxifene separate layer and the vitamin D separate layer each in the form of granules or tablets may be in the state of separation from one another, not intermingled with each other, in the composite capsule. In some embodiments, at least one of the raloxifene separate layer and the vitamin D separate layer is in the form of tablets. When at least one of the raloxifene separate layer and the vitamin D separate layer is in the form of tablets, the raloxifene separate layer and the vitamin D separate layer may be more completely separated from one another in the composite capsule, not intermingled with each other. In some other embodiments, the raloxifene separate layer and the vitamin D separate layer may be both in the form of tablets.

The raloxifene separate layer and the vitamin D separate layer may each independently further include a pharmaceutically acceptable additive, for example, a pharmaceutically acceptable additive for preparing granules or tablets. The pharmaceutically acceptable additive may be selected from a diluting agent, a disintegrating agent, a binding agent, a stabilizing agent, a lubricating agent, a coloring agent, and any combinations thereof, but is not limited thereto.

The diluting agent may be selected from the group consisting of microcrystalline cellulose, lactose, Rudy press, mannitol, calcium dihydrogen phosphate, starch, low-substituted hydroxypropyl cellulose, and any combinations thereof, but is not limited thereto. The amount of the diluting agent may be from about 1 wt % to about 99 wt %, and in some embodiments, about 5 wt % to about 90 wt %, based on a total weight of the granules or tablets

The disintegrating agent may be any disintegrating agents available in preparing granules or tablets. For example, the disintegrating agent may be selected from the groups consisting of crospovidone, pregelatinized starch, corn starch, methyl cellulose, hydroxypropyl methylcellulose (HPMC), sodium starch glycolate, croscarmellose sodium, low-substituted hydroxypropyl cellulose, starch, alginic acid or a sodium salt thereof, and any combinations thereof, but is not limited thereto. The amount of the disintegrating agent may be from about 1 wt % to about 35 wt % based on a total weight of the granules or tablets.

In some embodiments, the disintegrating agent may be a disintegrating agent not containing sodium ions (i.e., a sodium ion-noncontaining disintegrating agent). When the disintegrating agent is a sodium ion-containing disintegrating agent, the sodium ions of the disintegrating agent may inhibit dissolution of raloxifene by reaction with raloxifene.

In some embodiments, the sodium ion-noncontaining disintegrating agent may be a non-ionic disintegrating agent.

According to a result of a stability test under accelerated storage conditions, when a tablet or granules of raloxifene or vitamin D or a derivative thereof in the composite capsule according to an embodiment includes a sodium ion-noncontaining disintegrating agent, the amount of related (impurity) compounds of raloxifene were remarkably smaller than when using a sodium ion-containing disintegrating agent (for example, sodium starch glycolate, croscarmellose sodium, or sodium alginate) (refer to Test Example 2). Therefore, the composite capsule according to an embodiment was found to have further ensured stability of active ingredients when including a sodium ion-noncontaining disintegrating agent. The sodium ion-noncontaining disintegrating agent may be any known disintegrating agents available in preparing granules or tablets in the art. For example, the sodium ion-noncontaining disintegrating agent may be a non-ionic disintegrating agent selected from the group consisting of crospovidone, low-substituted hydroxypropyl cellulose, pregelatinized starch, corn starch, methyl cellulose, hydroxypropyl methylcellulose, alginic acid, and any combinations thereof.

As a test result, sodium ion-containing ionic disintegrating agents were found to have a negative effect on the dissolution of the composite capsule according to an embodiment. According to a result of a dissolution test (refer to Test Example 3), when the composite formulation according to an embodiment includes a sodium ion-noncontaining disintegrating agent, there was no effect on the dissolution of raloxifene. However, using a sodium ion-containing disintegrating agent was found to inhibit the dissolution of raloxifene in the composite capsule according to an embodiment. This is considered to be attributed to the fact that the dissolution rate of raloxifene may be reduced by the precipitation of crystals resulting from interaction between raloxifene and sodium ions.

In some embodiments, when tablets or granules of raloxifene or vitamin D or a derivative thereof in the composite capsules includes a sodium ion-noncontaining disintegrating agent, the composite capsule may have remarkably improved dissolution rate and stability of raloxifene, compared to when using a sodium ion-containing disintegrating agent.

The binding agent may be selected from the group consisting of hydroxypropylcellulose, hydroxypropyl methyl cellulose, polyvinylpyrrolidone, copovidone, macrogol, light anhydrous silicic acid, synthetic aluminum silicate, silicate derivatives such as calcium silicate or magnesium metasilicate aluminate, phosphate salts such as calcium hydrogen phosphate, carbonate salts such as calcium carbonate, and any combinations thereof, but is not limited thereto. The amount of the binding agent may be from about 1 wt % to about 30 wt %, and in some embodiments, about 2 wt % to about 15 wt %, based on a total weight of the granules or tablets.

The stabilizing agent may be an antioxidant, an acidifying agent, or a alkalizing agent. The antioxidant may be selected from butylated hydroxytoluene (BHT), butylated hydroxyanisole (BHA), ascorbic acid, ascorbyl palmitate, ethylenediaminetetraacetic acid (EDTA), sodium pyrosulfite, and any combinations thereof. For example, the antioxidant may be butylated hydroxytoluene. The acidifying agent may be selected from organic acids such as fumaric acid, citric acid, tartaric acid, succinic acid, lactic acid, malic acid, tosylic acid, oxalic acid, ascorbic acid, glutamic acid, alginic acid, maleic acid, adipic acid and the like; inorganic acids such as hydrochloric acid, sulfuric acid, nitric acid, phosphoric acid, acetic acid, boric acid, and the like; and any combinations thereof, but is not limited thereto. For example, the acidifying agent may be selected from fumaric acid, citric acid, tartaric acid, phosphoric acid and any combinations thereof.

The alkalizing agent may be selected from basic minerals such as sodium bicarbonate (NaHCO₃), calcium carbonate (CaCO₃), magnesium carbonate (MgCO₃), potassium dihydrogen phosphate (KH₂PO₄), potassium monohydrogen phosphate (K₂HPO₃), tribasic calcium phosphate, and the like; arginine, lysine, histidine, meglumine, aluminum magnesium silicate, aluminum magnesium metasilicate; and any combinations thereof, but is not limited thereto. For example, the alkalizing agent may be selected from sodium bicarbonate, calcium carbonate, magnesium carbonate, and any combinations thereof.

The stabilizing agent may be selected to be appropriate for the characteristics of the active pharmaceutical ingredients in the separate layers. The amount of the stabilizing agent may be from about 0.01 wt % to about 10 wt % based on a total weight of the active pharmaceutical ingredient.

The lubricating agent may be selected from the group consisting of stearic acid, metal salts of stearic acid such as calcium stearate or magnesium stearate, talc, colloid silica, sucrose fatty acid ester, hydrogenated vegetable oil, high-melting point wax, glyceryl fatty acid esters, glycerol dibehenate, and any combinations thereof, but is not limited thereto. The amount of the lubricating agent may be from about 0.2 wt % to about 5 wt %, and in some embodiments, about 0.3 wt % to about 3 wt %, based on a total weight of the tablets.

The tablets or granules may each independently further include a coating layer on a surface thereof. The coating layer may more completely separate the raloxifene separate layer and the vitamin D separate layer from one another not to be intermingled with each other. The amount of the coating layer may be from about 1 wt % to about 20 wt % based on a total weight of the granules or tablets. In some embodiments, at least one of the raloxifene separate layer and the vitamin D separate layer may be in the form of tablets, and the tablets may further include a coating layer on a surface thereof.

A coating material for the coating layer which may be on a surface of the granules or tablets may be a polymer known to be available for film coating in the field of granules or tablets. For example, the coating material may be selected from methyl cellulose, ethyl cellulose, polyvinyl alcohol, polyvinylpyrrolidone, hydroxyethyl cellulose, hydroxypropyl methyl cellulose, and any combinations thereof, but is not limited thereto. The amount of the coating material may be kept at minimal in consideration of the size of the formulation and preparation efficiency, but to maintain the function of the coating layer. The amount of the coating material may be from about 1 wt % to about 20 wt %, and in some embodiments, from about 2 wt % to about 10 wt %, based on a total weight of the granules or tablets.

The coloring agent may be selected from the group consisting of red ferric oxide, yellow ferric oxide, titanium oxide, Blue No. 1, Blue No. 2, and any combinations thereof, but is not limited thereto. The amount of the coloring agent may be from about 0.001 wt % to about 2 wt %, and in some embodiments, from about 0.01 wt % to about 1.5 wt %, based on a total weight of the granules or tablets.

In some embodiments, an empty capsule for the composite capsule may be a hard capsule. The hard capsule may be any hard capsules that are used in general in the preparation of medicine. For example, a capsule material of the hard capsule may be selected from gelatin, hypromellose, pullulan (NP Caps™, etc; available from Capsugel), polyvinyl alcohol, and any combinations thereof, but is not limited thereto.

The hard capsule may have any capsule sizes that are generally used in medicine. Commercially available capsules are numbered differently depending on the sizes thereof. For example, capsules of No. 00 (having a capsule cap diameter of about 8.5 mm and a capsule length of about 23.3 mm) may be so large that the elderly or patients of small build such as kids may feel uncomfortable with taking such a large capsule. Due to the large volume of such capsules, it may also be inconvenient to carry such large capsules. In some embodiments, an empty capsule for the composite capsule may be a capsule of No. 0, 1, 2, 3, or 4, due to mass limit of the tablets or granules to be in the capsule. For example, an empty capsule for the composite capsule may be a capsule of No. 1, 2, or 3.

The raloxifene separate layer of the composite capsule may include raloxifene or a pharmaceutically acceptable salt thereof. The pharmaceutically acceptable salt of raloxifene is known in the art, as disclosed in, for example, GB 2293382 and DE 19534744. Non-limiting examples of the pharmaceutically acceptable salt of raloxifene are pharmaceutically acceptable non-toxic organic acids such as acetic acid, citric acid, maleic acid, succinic acid, ascorbic acid, hydrochloric acid, hydrobromic acid, sulfuric acid and phosphoric acid; or inorganic addition salts. In some embodiments, the raloxifene separate layer may include raloxifene hydrochloride.

The daily dose of the raloxifene or a pharmaceutically acceptable salt thereof may be, on a 60-kg adult basis, about 10 mg to about 1,000 mg, and in some embodiments, about 30 mg to about 300 mg, and in some other embodiments, about 60 mg to about 80 mg. The daily dose of the raloxifene or a pharmaceutically acceptable salt thereof may vary depending on the race, ethnic group, age, gender, disease conditions of a patient, or the like, and may be appropriately increased or reduced by a specialist doctor in the art. In some embodiments, the composite capsule may include about 60 mg to about 80 mg of raloxifene or a pharmaceutically acceptable salt thereof per single unit dosage form.

The vitamin D separate layer of the composite capsule may include vitamin D or a derivative thereof. As used herein, the term “vitamin D or a derivative thereof” may refer to any types of vitamin D or derivatives thereof that are currently known to be 30o effective for the treatment of osteoporosis, for example, through bone mineral density improvement, or those that will be developed in the future. In some embodiments, the vitamin D or a derivative thereof may be cholecalciferol. The daily dose of cholecalciferol may be, on a 60-kg adult basis, about 200 IU to about 2,000 IU, and in some embodiments, about 400 IU to about 1,000 IU. The daily dose of cholecalciferol may vary depending on the race, ethnic group, age, gender, disease conditions of a patient, or the like, and may be appropriately increased or reduced by a specialist doctor in the art. In some embodiments, the composite capsule may include about 400 IU to about 1,000 IU of cholecalciferol per single unit dosage form.

According to embodiments, the composite capsule including raloxifene, which is known as a selective estrogen receptor modulator (SERM) drug, as a first active ingredient, and vitamin D or a derivative thereof as a second active ingredient may be used for the treatment of any diseases known for which co-administration of raloxifene with vitamin D or a derivative thereof is efficacious, and for the treatment of any future diseases not identified yet but for which co-administration of raloxifene with vitamin D or a derivative thereof may be efficacious. As used herein, the expression “treatment” may refer to the meaning of “treatment”, “improvement”, “amelioration”, and “management” of a disease. In some embodiments, the composite capsule may be used for bone mineral density improvement or for the treatment or prevention of vertebral fracture, non-vertebral fracture, osteoporosis, or non-invasive breast cancer.

In some embodiments, the composite capsule may be orally, buccally, or sublingually administrated. For example, the composite capsule may be orally administrated.

The composite capsule according to any of the embodiments may be constantly used for the prevention or treatment of osteoporosis. Due to the inclusion of the two active ingredients effective for the prevention or treatment of osteoporosis in a single unit dosage form, the composite capsule may remarkably improve compliance of a patient with osteoporosis that requires consistent administration of the two active ingredients. Due to the inclusion of raloxifene having less side effects and high safety and vitamin D or a derivative thereof that is effective for non-vertebral fracture in a single unit dosage form, the composite capsule may be used as an osteoporosis drug with good efficacy available even for the treatment of non-vertebral fracture. Furthermore, the composite capsule as a composite formulation including two active ingredients may have stable efficacy without stability reduction with time caused by the interaction between the two active ingredients during a preparation process. As a result of a comparison test, composite capsules according to embodiments of the present disclosure (Examples 1 to 5) were found to be remarkably higher in dissolution rate and stability with time, compared with the form of double-layered tablets that are widely used in preparing a composite formulation of plural active ingredients to block drug interaction (Comparative Example 3), tablets including a simple mixture of active ingredients (Comparative Example 1), and capsules including tablets of a simple mixture of active ingredients (Comparative Example 2). In other words, the composite capsule according to any of the above-described embodiments may have remarkably increased stability, compared to double-layered tablets that have been widely used as a type of formulation to separate active ingredients from one another and block interaction between the active ingredients, and thus may be suitable as a composite formulation of raloxifene, and vitamin D or a derivative thereof.

The composite capsule according to any of the above-described embodiments may include raloxifene, and vitamin D or a derivative thereof in a capsule, the two active ingredients completely separated from one another. Accordingly, reduced interactivity between the active ingredients may improve drug stability with time and consequentially therapeutic effects of the drug. The stability of the composite capsule according to any of the above-described embodiments with respect to time may also be evaluated using a common analysis method for a single formulation, without need for the development of an analysis method for the composite capsule.

In some embodiments, the composite capsule may be a composite capsule including a tablet of raloxifene or a pharmaceutically acceptable salt thereof and a tablet of vitamin D or a derivative thereof in a hard capsule.

In some embodiments, the composite capsule may be a composite capsule including a granule(s) of raloxifene or a pharmaceutically acceptable salt thereof and a tablet of vitamin D or a derivative thereof in a hard capsule.

In some other embodiments, the composite capsule may be a composite capsule including a tablet of raloxifene or a pharmaceutically acceptable salt thereof and a granule(s) of vitamin D or a derivative thereof in a hard capsule.

According to another aspect of the present disclosure, a method of preparing a composite capsule according to any of the above-described embodiments includes:

forming a mixture including raloxifene or a pharmaceutically acceptable thereof, and a pharmaceutically acceptable additive into granules or tablets;

forming a mixture including vitamin D or a derivative thereof, and a pharmaceutically acceptable additive into granules or tablets; and

filling a hard capsule with the granules or tablets of raloxifene and the granules or tablets of vitamin D or a derivative thereof to form a raloxifene separate layer and a vitamin D separated layer.

The above-detailed description of the composite capsule according to any of the above-described embodiments may apply to description of the method of preparing the composite capsule.

In some embodiments, in the method of preparing the composite capsule, the forming of a tablet may be performed using direct or indirect tableting. A direct tableting method may include mixing the active ingredient with a pharmaceutically acceptable additive to obtain a mixture and tableting the mixture. An indirect tableting method may include forming granules of the mixture and tableting the granules. The tableting of the mixture or granules may be performed using a tableting machine according to a general tableting method. The tablet may have an appropriate hardness, for example, about 1 kp to 30 kp. The hardness of tablets may be measured before formation of a film coating layer thereon.

In some embodiments, the method of preparing the composite granule may further include coating a surface of the granules or tablet before the filling of the hard capsule with the granules or tablet.

A vitamin D separate layer may include an increased amount of a related (impurity) compound due to the instability of vitamin D or a derivative thereof used as an active ingredient to water. Thus, preparing the vitamin D separate layer, for example, the forming of granules or tablet for the vitamin D separate layer, may be performed without using water or under the conditions including substantially no water.

The preparation method according to any embodiments does not need a special tableting equipment for producing double-layered tablets, and uses common methods of preparing granules, tablets, and/or capsules, and thus may be advantageous in economical aspects.

### MODE OF THE INVENTION

One or more embodiments of the present disclosure will now be described in detail with reference to the following examples. However, these examples are only for illustrative purposes and are not intended to limit the scope of the one or more embodiments of the present disclosure.

### Example 1: Preparation I of Composite Formulation

Povidone K30 and Polysorbate 80 as in Table 1 were dissolved in ethanol and distilled water to obtain a binder solution. The other ingredients of Table 1 were mixed to together and then wet-granulated with the binder solution, followed by sieving with a sieve having a mesh size of 30, and drying to obtain dried granules. The resulting dried raloxifine granules were tableted with a circular punch having a diameter of about 5.5 mm to prepare raloxifene tablets. Next, Opadry White was dissolved in distilled water and ethanol to prepare a coating solution, followed by coating the raloxifene tablets with the coating solution.

The ingredients for a cholecalciferol-containing layer of Table 2 were mixed together, followed by tableting to obtain cholecalciferol tablets. Opadry White and Blue No. 2 coloring agent were dissolved in distilled water and ethanol to obtain a coating solution, followed by coating the cholecalciferol tablets with the coating solution.

TABLE 1
raloxifene-containing layer

| Additives | Amounts (mg) |
|---|---|
| raloxifene hydrochloride | 30.00 |
| low-substituted hydroxypropyl cellulose | 26.60 |
| lactose hydrate | 16.40 |
| crospovidone | 11.60 |
| Povidone K30 | 1.60 |
| Polysorbate 80 | 1.20 |
| light anhydrous silicic acid | 1.60 |
| magnesium stearate | 1.00 |
| Opadry White | 4.50 |
| distilled water | (36.00) |
| ethanol | (12.00) |

TABLE 2
cholecalciferol-containing layer

| Additives | Amounts (mg) |
|---|---|
| concentrated cholecalciferol powder (109,000 IU/g) | 7.34 |
| lactose hydrate | 61.26 |
| microcrystalline cellulose | 10.00 |
| crospovidone | 5.00 |
| light anhydrous silicic acid | 0.50 |
| magnesium stearate | 0.90 |
| Opadry White | 2.50 |
| Blue No. 2 | Appropriate amount |
| distilled water | (15.00) |
| ethanol | (5.00) |

Two of the raloxifene tablets and one of the cholecalciferol tablets were filled into a hard capsule (No. 1) including gelatin as a main material to prepare a composite capsule including 60 mg of raloxifene and 800 IU of cholecalciferol. FIG. 1 is a schematic view of this composite capsule.

### Example 2: Preparation II of Composite Formulation

A composite capsule including 60 mg of raloxifene and 800 IU of cholecalciferol were prepared in the same manner as in Example 1, except that a hard capsule including hypromellose as a main material was used.

### Example 3: Preparation III of Composite Formulation

A composite capsule including 60 mg of raloxifene and 800 IU of cholecalciferol were prepared in the same manner as in Example 1, except that a hard capsule including pullulan as a main material was used.

### Example 4: Preparation IV of Composite Formulation

Povidone K30 and Polysorbate 80 as in Table 3 were dissolved in ethanol and distilled water to obtain a binder solution. The other ingredients of Table 3 were mixed together and then wet-granulated with the binder solution, followed by sieving with a sieve having a mesh size of 30, and drying to obtain raloxifine granules.

Cholecalciferol tablets according to the composition of Table 4 were prepared in the same manner as in Example 1.

TABLE 3
raloxifene-containing layer

| Additives | Amounts (mg) |
|---|---|
| raloxifene hydrochloride | 60.00 |
| low-substituted hydroxypropyl cellulose | 53.20 |
| lactose hydrate | 32.80 |
| crospovidone | 23.20 |
| Povidone K30 | 3.20 |
| Polysorbate 80 | 2.40 |
| light anhydrous silicic acid | 3.20 |
| magnesium stearate | 2.00 |

TABLE 4
cholecalciferol-containing layer

| Additives | Amounts (mg) |
|---|---|
| concentrated cholecalciferol powder (109,000 IU/g) | 7.34 |
| lactose hydrate | 61.26 |
| microcrystalline cellulose | 10.00 |
| crospovidone | 5.00 |
| light anhydrous silicic acid | 0.50 |
| magnesium stearate | 0.90 |
| Opadry White | 2.50 |
| Blue No. 2 | Appropriate amount |
| distilled water | (15.00) |
| ethanol | (5.00) |

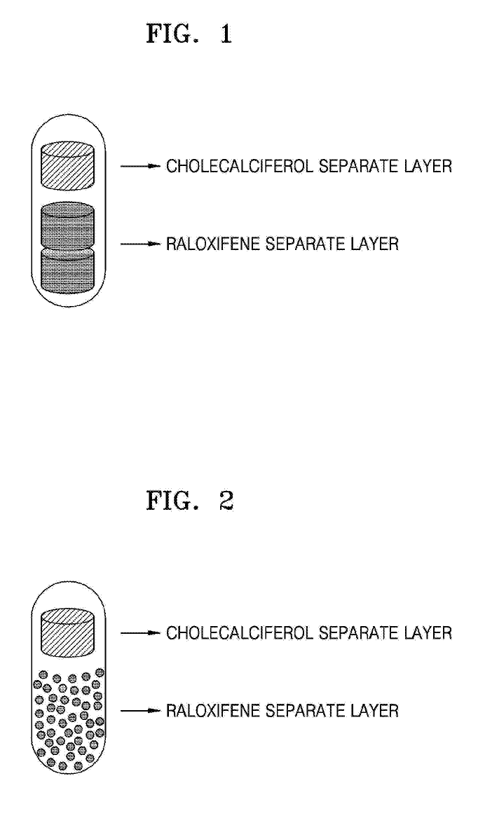

The raloxifene granules and one of the cholecalciferol tablets were filled into a hard capsule (No. 1) including gelatin as a main material to prepare a composite capsule including 60 mg of raloxifene and 800 IU of cholecalciferol. FIG. 2 is a schematic view of this composite capsule.

### Example 5: Preparation V of Composite Formulation

Raloxifene tablets according to the composition of Table 5 were prepared in the same manner as in Example 1.

Concentrated cholecalciferol powder and hydroxypropylcellulose as in Table 6 were dissolved in distilled water to obtain a binder solution. The other ingredients of Table 6 were mixed together and then wet-granulated with the binder solution, followed by sieving with a sieve having a mesh size of 30, and drying to obtain cholecalciferol granules.

TABLE 5
raloxifene-containing layer

| Additives | Amounts (mg) |
|---|---|
| raloxifene hydrochloride | 30.00 |
| low-substituted hydroxypropyl cellulose | 26.60 |
| lactose hydrate | 16.40 |
| crospovidone | 11.60 |
| Povidone K30 | 1.60 |
| Polysorbate 80 | 1.20 |
| light anhydrous silicic acid | 1.60 |
| magnesium stearate | 1.00 |
| Opadry White | 4.50 |
| distilled water | (36.00) |
| ethanol | (12.00) |

TABLE 6
cholecalciferol-containing layer

| Additives | Amounts (mg) |
|---|---|
| concentrated cholecalciferol powder (109,000 IU/g) | 7.34 |
| lactose hydrate | 60.16 |
| microcrystalline cellulose | 10.00 |
| hydroxypropylcellulose | 4.00 |
| crospovidone | 3.00 |
| light anhydrous silicic acid | 0.50 |

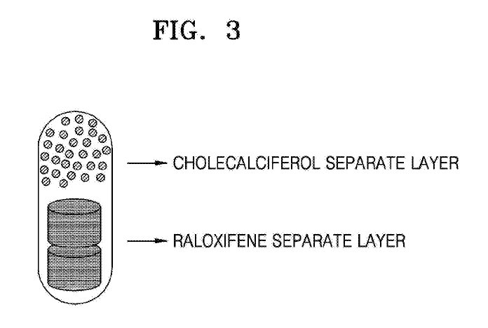

Two of the raloxifene tablets and the cholecalciferol granules were filled into a hard capsule (No. 1) including gelatin as a main material to prepare a composite capsule including 60 mg of raloxifene and 800 IU of cholecalciferol. FIG. 3 is a schematic view of this composite capsule.

### Examples 6 to 8: Preparation VI of Composite Formulation

Composite capsules were prepared in the same manner as in Example 1, except that raloxifene tablets and cholecalciferol tablets were prepared according to the compositions of Tables 7 to 9.

TABLE 7
Example 6

| raloxifene-containing layer | | cholecalciferol-containing layer | |
|---|---|---|---|
| | Amounts | | Amounts |
| Additives | (mg) | Additives | (mg) |
| raloxifene hydrochloride | 30 | concentrated | 7.34 |
| | | cholecalciferol | |
| | | powder | |
| | | (109,000 IU/g) | |
| low-substituted | 26.6 | lactose hydrate | 61.26 |
| hydroxypropyl cellulose | | | |
| lactose hydrate | 16.4 | microcrystalline | 10 |
| | | cellulose | |
| sodium starch glycolate | 11.6 | crospovidone | 5 |
| Povidone K30 | 1.6 | light anhydrous | 0.5 |
| | | silicic acid | |
| Polysorbate 80 | 1.2 | magnesium | 0.9 |
| | | stearate | |
| light anhydrous silicic acid | 1.6 | Opadry White | 2.5 |
| magnesium stearate | 1 | Blue No. 2 | Appropriate |
| | | | amount |
| Opadry White | 4.5 | distilled water | (15) |
| distilled water | (36) | ethanol | (5) |
| ethanol | (12) | | |

TABLE 8
Example 7

| raloxifene-containing layer | | cholecalciferol-containing layer | |
|---|---|---|---|
| | Amounts | | Amounts |
| Additives | (mg) | Additives | (mg) |
| raloxifene hydrochloride | 30 | concentrated | 7.34 |
| | | cholecalciferol | |
| | | powder | |
| | | (109,000 IU/g) | |
| low-substituted | 26.6 | lactose hydrate | 61.26 |
| hydroxypropyl cellulose | | | |
| lactose hydrate | 16.4 | microcrystalline | 10 |
| | | cellulose | |
| croscarmellose sodium | 11.6 | crospovidone | 5 |
| Povidone K30 | 1.6 | light anhydrous | 0.5 |
| | | silicic acid | |
| Polysorbate 80 | 1.2 | magnesium | 0.9 |
| | | stearate | |
| light anhydrous silicic acid | 1.6 | Opadry White | 2.5 |
| magnesium stearate | 1 | Blue No. 2 | Appropriate |
| | | | amount |
| Opadry White | 4.5 | distilled water | (15) |
| distilled water | (36) | ethanol | (5) |
| ethanol | (12) | | |

TABLE 9
Example 8

| raloxifene-containing layer | | cholecalciferol-containing layer | |
|---|---|---|---|
| | Amounts | | Amounts |
| Additives | (mg) | Additives | (mg) |
| raloxifene hydrochloride | 30 | concentrated | 7.34 |
| | | cholecalciferol | |
| | | powder | |
| | | (109,000 IU/g) | |
| low-substituted | 26.6 | lactose hydrate | 61.26 |
| hydroxypropyl cellulose | | | |
| lactose hydrate | 16.4 | microcrystalline | 10 |
| | | cellulose | |
| sodium alginate | 11.6 | crospovidone | 5 |
| Povidone K30 | 1.6 | light anhydrous | 0.5 |
| | | silicic acid | |
| Polysorbate 80 | 1.2 | magnesium | 0.9 |
| | | stearate | |
| light anhydrous silicic acid | 1.6 | Opadry White | 2.5 |
| magnesium stearate | 1 | Blue No. 2 | Appropriate |
| | | | amount |
| Opadry White | 4.5 | distilled water | (15) |
| distilled water | (36) | ethanol | (5) |
| ethanol | (12) | | |

### Comparative Example 1: Preparation VII of Composite Formulation (as a Tablet Including a Simile Mixture of Active Ingredients)

Povidone K30 and Polysorbate 80 as in Table 10 were dissolved in ethanol and distilled water to obtain a binder solution. Raloxifene, cholecalciferol, hydroxypropyl methylcellulose, lactose anhydrous, and crospovidone as in Table 10 were mixed together and then wet-granulated with the binder solution, followed by sieving with a sieve having a mesh size of 30, and drying to obtain dried granules. The resulting dried granules were mixed with light anhydrous silicic acid and magnesium stearate as in Table 10 and then tableted to prepare a tablet. Next, Opadry White was dissolved in distilled water and ethanol to prepare a coating solution, followed by coating the tablet with the coating solution to prepare a composite tablet including 60 mg of raloxifene and 800 IU of cholecalciferol.

TABLE 10

| Additives | Amounts (mg) |
|---|---|
| raloxifene hydrochloride | 60.00 |
| concentrated cholecalciferol powder (109,000 IU/g) | 7.34 |
| low-substituted hydroxypropyl cellulose | 53.20 |
| lactose hydrate | 32.80 |
| crospovidone | 23.20 |
| Povidone K30 | 3.20 |
| Polysorbate 80 | 2.40 |
| light anhydrous silicic acid | 3.20 |
| magnesium stearate | 2.00 |
| Opadry White | 9.00 |
| distilled water | (72.00) |
| ethanol | (24.00) |

### Comparative Example 2: Preparation VIII of Composite Formulation (as a Capsule Including a Tablet of Simple Mixture of Active Ingredients)

Composite tablets according to the composition of Table 11 were prepared in the same manner as in Comparative Example 1, except that a hard capsule including gelatin as a main material was filled with two of the composite tablets to prepare a capsule including 60 mg of raloxifene and 800 IU of cholecalciferol.

TABLE 11

| Additives | Amounts (mg) |
|---|---|
| raloxifene hydrochloride | 30.00 |
| concentrated cholecalciferol powder (109,000 IU/g) | 3.67 |
| low-substituted hydroxypropyl cellulose | 26.6 |
| lactose hydrate | 16.4 |
| crospovidone | 11.6 |
| Povidone K30 | 1.60 |
| Polysorbate 80 | 1.20 |
| light anhydrous silicic acid | 1.60 |
| magnesium stearate | 1.00 |
| Opadry White | 4.50 |
| distilled water | (36.00) |
| ethanol | (12.00) |

### Comparative Example 3: Preparation IX of Composite Formulation (as a Double-Layered Tablet)

According to the composition for a raloxifene-containing layer in Table 12, Povidone K30 and Polysorbate 80 were dissolved in ethanol and distilled water to obtain a binder solution. Raloxifene hydrochloride, low-substituted hydroxypropyl cellulose, crospovidone as in Table 12 were mixed together and then wet-granulated with the binder solution, followed by sieving with a sieve having a mesh size of 30, and drying to obtain dried granules. The resulting dried granules were mixed with lactose hydrate, light anhydrous silicic acid, and magnesium stearate as in Table 12 and then tableted to prepare a raloxifene tablet.

According to the composition for a cholecalciferol-containing layer in Table 12, cholecalciferol, lactose hydrate, microcrystalline cellulose, crospovidone, light anhydrous silicic acid, and magnesium stearate were mixed together and then tableted together with the raloxifene tablet, to thereby prepare a double-layered tablet.

Next, Opadry White was dissolved in distilled water and ethanol to prepare a coating solution, followed by coating the double-layered tablet with the coating solution to prepare a composite formulation including 60 mg of raloxifene and 800 IU of cholecalciferol.

TABLE 12

| raloxifene-containing layer | | cholecalciferol-containing layer | |
|---|---|---|---|
| | Amounts | | Amounts |
| Additives | (mg) | Additives | (mg) |
| raloxifene | 60.00 | concentrated | 7.34 |
| hydrochloride | | cholecalciferol powder | |
| | | (109,000 IU/g) | |
| low-substituted | 53.20 | lactose hydrate | 61.26 |
| hydroxypropyl | | | |
| cellulose | | | |
| lactose hydrate | 32.80 | microcrystalline cellulose | 10.00 |
| crospovidone | 23.20 | crospovidone | 5.00 |
| Povidone K30 | 3.20 | light anhydrous silicic acid | 0.50 |
| Polysorbate 80 | 2.40 | magnesium stearate | 0.90 |
| light anhydrous | 3.20 | Opadry White | 4.50 |
| silicic acid | | | |
| magnesium stearate | 2.00 | distilled water | (36.00) |
| | | ethanol | (12.00) |

### Test Example 1: Dissolution Test on Composite Formulations

A dissolution test on the composite formulations of Examples 1 to 5 and Comparative Examples 1 to 3 was performed according to the recommended dissolution test method of the Food and Drug Administration.

About 1,000 mL of a 0.1% Polysorbate 80 solution was used as a dissolution medium for raloxifene. The dissolution test method used for raloxifene was the paddle method, the temperature of the dissolution medium was about 37±5° C., and the paddle speed was about 50 rpm. About 500 mL of a 0.3% sodium lauryl sulfate solution was used as a dissolution medium for cholecalciferol. The dissolution test method used for cholecalciferol was the paddle method, the temperature of the dissolution medium was about 37±5° C., and the paddle speed was about 75 rpm. About 3 mL of a sample was taken after 5 min, 10 min, 15 min, 20 min, 30 min, and 45 min from the start of the dissolution test, and the same volume of the dissolution medium was added each time for a constant total volume of the dissolution medium. Each sample solution from the dissolution test was filtered using a 0.45-μm membrane filter, and then analyzed by high-performance liquid chromatography under the conditions stated below to evaluate dissolution rates of raloxifene and cholecalciferol.

<Dissolution Test Analysis Conditions for Raloxifene>

Analysis equipment: HPLC (Hitachi 2000 series, Japan)

Detector: Ultraviolet ray (UV) absorbance detector (wavelength: 290 nm)

Column: Stainless steel column (Zorbax CN Column, having an inner diameter of about 4.6 mm and a length of about 15 cm) packed with particles (having a diameter of about 5 μm) of cyanated silicagel for liquid chromatography

Mobile phase: A solution prepared by mixing a mixture of 500 mL of acetonitrile and 500 mL of water with 2.0 mL of triethylamine and pH adjustment to 4.0 with phosphoric acid.

Flow rate: 2.0 mL/min

Column temperature: 30° C.

<Dissolution Test Analysis Conditions for Cholecalciferol>

Analysis equipment: HPLC (Hitachi 2000 series, Japan)

Detector: UV absorbance detector (wavelength: 265 nm)

Column: Stainless steel column (Platinum EPS C18 Column, having an inner diameter of about 4.6 mm and a length of about 15 cm) packed with particles (having a diameter of about 3 μm) of octadecyl silicagel for liquid chromatography

Mobile phase: Acetonitrile:water=93:7 (v/v)

Flow rate: 1.5 mL/min

Column temperature: 30° C.

The resulting dissolution rates of raloxifene and cholecalciferol analyzed under the above-described analysis conditions are shown in Tables 13 and 14.

TABLE 13

| | Raloxifene dissolution rate | | | | |
|---|---|---|---|---|---|
| Example | 5 min | 10 min | 15 min | 30 min | 45 min |
| Example 1 | 14.8% | 61.3% | 82.3% | 96.7% | 100.1% |
| Example 2 | 5.6% | 20.5% | 70.6% | 94.4% | 99.4% |
| Example 3 | 7.2% | 40.5% | 75.1% | 95.8% | 100.8% |
| Example 4 | 78.6% | 94.4% | 90.1% | 98.6% | 100.6% |
| Example 5 | 16.8% | 60.3% | 84.7% | 97.2% | 101.3% |
| Comparative | 20.4% | 65.8% | 85.1% | 97.1% | 100.1% |
| Example 1 | | | | | |
| Comparative | 10.8% | 59.3% | 80.4% | 95.3% | 99.8% |
| Example 2 | | | | | |
| Comparative | 25.1% | 71.8% | 86.6% | 96.3% | 101.7% |
| Example 3 | | | | | |

TABLE 14

| | Cholecalciferol dissolution rate | | | | |
|---|---|---|---|---|---|
| Example | 5 min | 10 min | 15 min | 30 min | 45 min |
| Example 1 | 20.8% | 79.0% | 98.6% | 98.8% | 99.9% |
| Example 2 | 6.4% | 69.2% | 96.4% | 97.9% | 100.0% |
| Example 3 | 9.3% | 75.1% | 99.1% | 99.1% | 100.3% |
| Example 4 | 19.7% | 77.7% | 98.5% | 100.5% | 100.2% |
| Example 5 | 82.6% | 92.4% | 99.6% | 99.4% | 99.6% |
| Comparative | 17.6% | 48.9% | 72.8% | 81.6% | 86.2% |
| Example 1 | | | | | |
| Comparative | 16.2% | 45.6% | 75.6% | 86.6% | 85.9% |
| Example 2 | | | | | |
| Comparative | 21.9% | 50.7% | 80.6% | 84.9% | 85.6% |
| Example 3 | | | | | |

Referring to the dissolution test results of Table 13, the composite formulations of Examples 1 to 5 and Comparative Examples 3 were found to have nearly similar raloxifene dissolution rates after 15 min. However, referring to the dissolution test results of Table 14, the composite formulations of Examples 1 to 5 were found to have cholecalciferol dissolution rates of about 95% or higher after 15 min and of nearly 100% after 45 min, while the composite formulations of Comparative Examples 1 to 3 had a cholecalciferol dissolution rate of about 80% or less after 15 min and of about 85% even after 45 min. Such low cholecalciferol dissolution rate of the composite formulations of Comparative Examples 1 to 3 is considered to be attributed to reduced stability of the active ingredient. Therefore, composite capsules according to embodiments are found as remarkably effective formulations with improved dissolution rate and stability, compared to other types of composite formulations.

### Test Example 2: Accelerated Storage Stability Test on Composite Formulations

A stability test on the composite formulations of raloxifene and cholecalciferol according to Examples 1 to 8 and Comparative Examples 1 to 3 was performed after storage under the following accelerated storage conditions by analyzing the levels of related (impurity) compounds resulting from raloxifene and cholecalciferol under the following conditions.

<Accelerated Storage Conditions>

Storage conditions: storage in high-density polyethylene (HDPE) bottle at 40□ and a relative humidity of about 75%

Testing time: Initial (before storage), and after storage for 1 month, 3 months, and 6 months

Target analyte: Raloxifene and cholecalciferol

<Analysis Condition for Raloxifene and Raloxifene-Related Compounds>

Analysis equipment: HPLC (Hitachi 2000 series, Japan)

Detector: UV absorbance detector (wavelength: 280 nm)

Column: Stainless steel column (Inertsil C8 Column, having an inner diameter of about 4.6 mm and a length of about 25 cm) packed with particles (having a diameter of about 5 μm) of silicagel for liquid chromatography

Mobile phase A: Phosphate buffer solution (pH 3.0): acetonitrile=75:25

Mobile phase B: Phosphate buffer solution (pH 3.0): acetonitrile=50:50

Phosphate buffer solution (pH 3.0): A solution prepared by dissolving 9.0 g of anhydrous potassium dihydrogen phosphate in about 1 L of water, adding 0.5 mL of phosphoric acid thereto, and pH adjustment to 3.0 with a phosphoric acid or potassium hydroxide solution.

Mobile phase gradient conditions: see Table 15

TABLE 15

| Time (min) | Mobile phase A (%) | Mobile phase B (%) |
|---|---|---|
| 0 | 100 | 0 |
| 5 | 100 | 0 |
| 36.25 | 0 | 100 |
| 38.25 | 100 | 0 |
| 48 | 100 | 0 |

Flow rate: 1.0 mL/min

Column temperature: 350

<Analysis Condition for Cholecalciferol and Cholecalciferol-Related Compounds>

Analysis equipment: HPLC (Hitachi 2000 series, Japan)

Detector: UV absorbance detector (wavelength: 265 nm)

Column: Stainless steel column (Phenosphere ODS Column, having an inner diameter of about 4.6 mm and a length of about 15 cm) packed with particles (having a diameter of about 3 μm) of octadecyl silicagel for liquid chromatography

Mobile phase A: 0.025% aqueous solution of phosphoric acid

Mobile phase B: Mixed solution of acetonitrile and Mobile phase A of 99:1 (v/v)

Mobile phase gradient conditions: see Table 16

TABLE 16

| Time (min) | Mobile phase A (%) | Mobile phase B (%) |
|---|---|---|
| 0 | 51.5 | 48.5 |
| 16 | 13 | 87 |
| 39 | 10 | 90 |
| 43 | 0 | 100 |
| 57 | 0 | 100 |
| 57.1 | 51.5 | 48.5 |
| 65 | 51.5 | 48.5 |

Flow rate: 1.2 mL/min

Column temperature: 25° C.

The types and levels of the raloxifene-related compounds and cholecalciferol-related compounds analyzed under the above-described conditions are shown in Tables 17 and 18.

TABLE 17

| | Raloxifene-related compound | | | | | | | |
|---|---|---|---|---|---|---|---|---|
| | Initial | | 1 month | | 3 months | | 6 months | |
| | | Total | | Total | | Total | | Total |
| | | related | | related | | related | | related |
| Example | N-Oxide | compound | N-Oxide | compound | N-Oxide | compound | N-Oxide | compound |
| Example 1 | 0.00% | 0.04% | 0.03% | 0.07% | 0.04% | 0.10% | 0.07% | 0.20% |
| Example 2 | 0.01% | 0.05% | 0.02% | 0.06% | 0.05% | 0.12% | 0.06% | 0.21% |
| Example 3 | 0.01% | 0.05% | 0.03% | 0.08% | 0.05% | 0.11% | 0.08% | 0.19% |
| Example 4 | 0.00% | 0.05% | 0.02% | 0.06% | 0.05% | 0.12% | 0.06% | 0.21% |
| Example 5 | 0.01% | 0.05% | 0.04% | 0.09% | 0.08% | 0.16% | 0.10% | 0.35% |
| Example 6 | 0.02% | 0.06% | 0.06% | 0.17% | 0.13% | 0.29% | 0.21% | 0.52% |
| Example 7 | 0.01% | 0.05% | 0.08% | 0.20% | 0.16% | 0.33% | 0.20% | 0.49% |
| Example 8 | 0.01% | 0.05% | 0.05% | 0.19% | 0.14% | 0.34% | 0.18% | 0.48% |
| Comparative | 0.01% | 0.07% | 0.14% | 0.36% | 0.29% | 0.87% | 0.79% | 1.20% |
| Example 1 | | | | | | | | |
| Comparative | 0.00% | 0.04% | 0.15% | 0.34% | 0.31% | 0.80% | 0.72% | 1.18% |
| Example 2 | | | | | | | | |
| Comparative | 0.02% | 0.05% | 0.12% | 0.29% | 0.24% | 0.71% | 0.57% | 1.01% |
| Example 3 | | | | | | | | |

TABLE 18

| | Cholecalciferol-related compound | | | | | | | |
|---|---|---|---|---|---|---|---|---|
| | Initial | | 1 month | | 3 months | | 6 months | |
| | Trans- | Total | Trans- | Total | Trans- | Total | Trans- | Total |
| Example | VitD | esters | VitD | esters | VitD | esters | VitD | esters |
| Example 1 | 0.04% | 0.25% | 0.05% | 0.31% | 0.06% | 0.40% | 0.10% | 0.52% |
| Example 2 | 0.03% | 0.24% | 0.05% | 0.29% | 0.07% | 0.39% | 0.11% | 0.54% |
| Example 3 | 0.04% | 0.23% | 0.06% | 0.33% | 0.06% | 0.42% | 0.13% | 0.55% |
| Example 4 | 0.04% | 0.24% | 0.05% | 0.29% | 0.08% | 0.39% | 0.11% | 0.54% |
| Example 5 | 0.05% | 0.26% | 0.06% | 0.31% | 0.07% | 0.41% | 0.12% | 0.51% |
| Example 6 | 0.05% | 0.26% | 0.06% | 0.28% | 0.08% | 0.37% | 0.12% | 0.55% |
| Example 7 | 0.04% | 0.28% | 0.04% | 0.32% | 0.07% | 0.41% | 0.14% | 0.58% |
| Example 8 | 0.04% | 0.26% | 0.04% | 0.30% | 0.08% | 0.42% | 0.13% | 0.51% |
| Comparative | 0.03% | 0.27% | 0.23% | 0.61% | 0.50% | 0.79% | 0.74% | 1.59% |
| Example 1 | | | | | | | | |
| Comparative | 0.03% | 0.25% | 0.19% | 0.59% | 0.46% | 0.75% | 0.71% | 1.48% |
| Example 2 | | | | | | | | |
| Comparative | 0.04% | 0.24% | 0.20% | 0.55% | 0.48% | 0.71% | 0.65% | 1.29% |
| Example 3 | | | | | | | | |

Referring to Tables 17 and 18 as the results of related compound analysis, the composite capsules of raloxifene and cholecalciferol according to Examples 1 to 8 were found to have a small increase in the related compounds over time for 6 months under the accelerated conditions. However, the composite formulations of Comparative Examples 1 to 3 in the forms of a mixture tablet including a mixture of the active ingredients, a capsule including the mixture tablet, and a double-layered tablet, respectively, were found to have a remarkable increase in the related compounds at every testing analyte and time over 6 months, compared to the composite capsules of Examples 1 to 8. These results indicate that composite capsules according to embodiments including raloxifene and cholecalciferol as separate layers may be composite formulations with improved stabilities of both raloxifene and cholecalciferol.

In particular, the composite capsules of Examples 6 to 8 including a sodium ion-containing disintegrating agent were found to be similar in the types and levels of cholecalciferol-related compounds, compared to the composite capsules of Examples 1 to 5 including a sodium ion-noncontaining disintegrating agent, and to have a remarkable low level of raloxifene-related compounds, compared to the composite formulations of Comparative Examples 1 to 3, but a remarkable high level of raloxifene-related compounds, compared to the formulations capsules of Examples 1 to 5. Therefore, the composite capsules according to embodiments including a sodium ion-noncontaining disintegrating agent were found to have further improved stability of active ingredients, compared to when using a sodium ion-containing disintegrating agent.

### Test Example 3: Dissolution Test with Different Disintegrating Agents

A raloxifene dissolution test was performed on the composite capsules of Examples 1, 6, 7, and 8 in the same manner as in Test Example 1, according to the dissolution method recommended by the FDA.

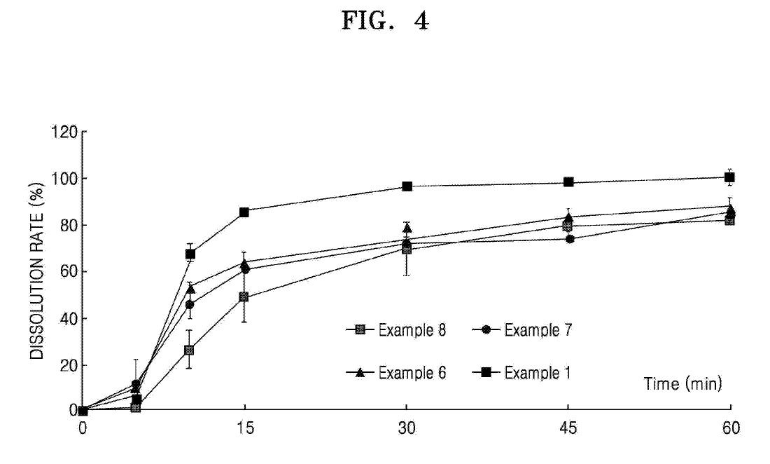

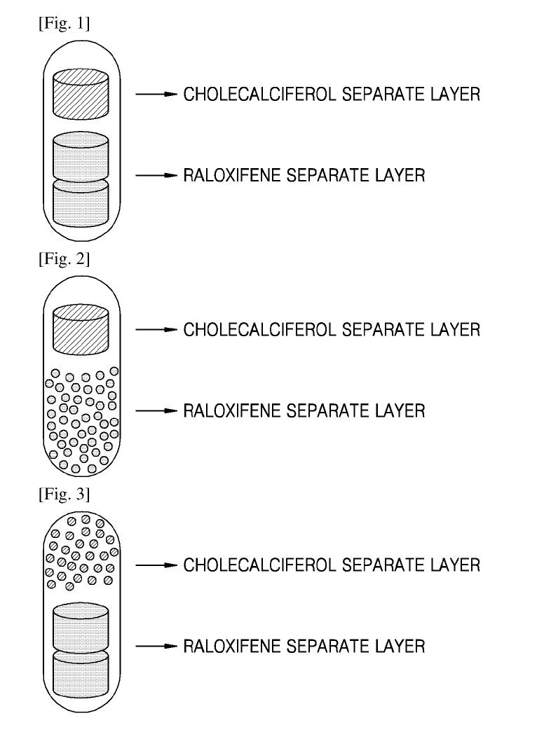

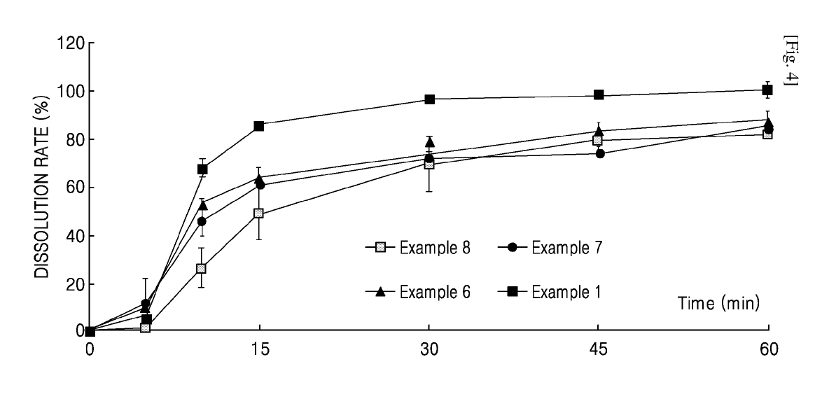

The results of the raloxifene dissolution test on the composite capsules of Examples 1, 6, 7, and 8 are shown in Table 19 and FIG. 4.

TABLE 19

| | Raloxifene dissolution rate (%) | | | | | | | |
|---|---|---|---|---|---|---|---|---|
| | Example 6 | | Example 7 | | Example 8 | | Example 1 | |
| Time | | Standard | | Standard | | Standard | | Standard |
| (min) | Average | deviation | Average | deviation | Average | deviation | Average | deviation |
| 0 | 0.0 | 0.0 | 0.0 | 0.0 | 0.0 | 0.0 | 0.0 | 0.0 |
| 5 | 10.5 | 11.6 | 11.9 | 2.3 | 1.1 | 0.3 | 5.5 | 4.7 |
| 10 | 53.3 | 2.3 | 46.4 | 7.0 | 26.5 | 8.0 | 68.5 | 3.8 |
| 15 | 64.5 | 3.9 | 61.5 | 2.4 | 48.9 | 10.5 | 86.3 | 1.9 |
| 30 | 79.2 | 0.1 | 72.2 | 2.9 | 69.8 | 11.7 | 97.5 | 2.5 |
| 45 | 84.1 | 3.0 | 74.6 | 2.4 | 79.4 | 5.5 | 98.6 | 1.9 |
| 60 | 88.0 | 3.7 | 85.1 | 0.7 | 82.3 | 4.1 | 100.7 | 1.3 |

Referring to Table 19 and FIG. 4, the composite capsule of Example 1 using a sodium ion-noncontaining disintegrating agent was found to have a remarkably higher dissolution rate, compared to the composite capsules of Examples 6 to 8 using a sodium ion-containing disintegrating agent. Therefore, sodium ion-containing disintegrating agents were found to inhibit the dissolution of raloxifene, which may be attributed to the fact that the dissolution rate of raloxifene may be reduced by the precipitation of crystals resulting from interaction between raloxifene and sodium ions.

While this invention has been particularly shown and described with reference to preferred embodiments thereof, it will be understood by those skilled in the art that various changes in form and details may be made therein without departing from the spirit and scope of the invention as defined by the appended claims. The disclosed embodiments should be considered in descriptive sense only and not for purposes of limitation. Therefore, the scope of the invention is defined not by the detailed description of the invention but by the appended claims, and all differences within the scope will be construed as being included in the present invention.

1. A composite capsule comprising:
a raloxifene separate layer comprising raloxifene or a pharmaceutically acceptable salt thereof; and
a vitamin D separate layer comprising vitamin D or a derivative thereof,
wherein the raloxifene separate layer and the vitamin D separate layer are separated from one another in the composite capsule.

2. The composite capsule of claim 1, wherein the raloxifene separate layer and the vitamin D separate layer are each in the form of granules or tablets.

3. The composite capsule of claim 2, wherein at least one of the raloxifene separate layer and the vitamin D separate layer is in the form of tablets.

4. The composite capsule of claim 1, wherein the capsule is made from a material selected from the group consisting of gelatin, hypromellose, pullulan, polyvinyl alcohol, and any combinations thereof.

5. The composite capsule of claim 1, wherein the pharmaceutically acceptable salt of raloxifene is raloxifene hydrochloride.

6. The composite capsule of claim 1, wherein the vitamin D or a derivative thereof is cholecalciferol.

7. The composite capsule of claim 2, wherein the raloxifene separate layer and the vitamin D separate layer each independently comprises a sodium ion-noncontaining disintegrating agent.

8. The composite capsule of claim 7, wherein the sodium ion-noncontaining disintegrating agent is a non-ionic disintegrating agent selected from the group consisting of crospovidone, low-substituted hydroxypropyl cellulose, pregelatinized starch, corn starch, methyl cellulose, hydroxypropyl methylcellulose, alginic acid, and any combinations thereof.

9. The composite capsule of claim 3, wherein at least one of the tablets further comprises a coating layer.

10. The composite capsule of claim 9, wherein the amount of the coating layer is from about 1 wt % to about 20 wt % based on a total weight of the tablet.

11. The composite capsule of claim 1, wherein the composite capsule comprises about 60 mg to about 80 mg of the raloxifene or a pharmaceutically acceptable salt thereof and about 400 IU to about 1,000 IU of the vitamin D or a derivative thereof per single unit dosage form.

12. The composite capsule of claim 1, wherein the composite capsule is for bone mineral density improvement or for the treatment or prevention of vertebral fracture, non-vertebral fracture, osteoporosis, or non-invasive breast cancer.

13. A method of preparing a composite capsule according to claim 12, the method comprising:
forming a mixture including raloxifene or a pharmaceutically acceptable thereof, and a pharmaceutically acceptable additive into granules or tablets;
forming a mixture including vitamin D or a derivative thereof, and a pharmaceutically acceptable additive into granules or tablets; and
filling a hard capsule with the granules or tablets of raloxifene and the granules or tablets of vitamin D or a derivative thereof to form a raloxifene separate layer and a vitamin D separated layer.

14. The method of claim 13, wherein the forming of the tablets of raloxifene or vitamin D further include coating the tablets.

15. The method of claim 13, wherein the forming of the granules or tablets of vitamin D or a derivative thereof substantially does not use water.
